(19) United States
(12) Patent Application Publication
(10) Pub. No.: US 2017/0281222 A1
(43) Pub. Date: Oct. 5, 2017

(54) Compound Curette with Soft Handle and Liquid Delivery System

(71) Applicant: Pamela Bissell, Kohler, WI (US)

(72) Inventor: Pamela Bissell, Kohler, WI (US)

(21) Appl. No.: 15/470,069

(22) Filed: Mar. 27, 2017

Related U.S. Application Data
(60) Provisional application No. 62/314,866, filed on Mar. 29, 2016.

(51) Int. Cl. A61B 17/3207; A61B 17/54

(52) U.S. Cl. CPC A61B 17/320708; A61B 17/54

(57) ABSTRACT

An improved curette for removing tissue samples and delivering liquid and anti-pathogen to the treated area is presented. Curette has a soft and squeezable reservoir-handle, allowing for easy maneuvering around the crevices of the treated area. The handle is filled with liquid and attached to a scraping end, that is connected to the handle via hollow tube. The scraping end of the curette is made of a metal or a hard plastic and could be coated in anti-pathogen material. The liquid delivery mechanism allows placing liquid directly to the treated area. The method of scraping debris and medication delivery is particularly useful for a treatment of the toenail fungus. Main portions of the curette could be detachable, allowing for the easy reservoir replacement and for the use of multiple scraping utensils. Similarly, the device could be manufactured for a single use when all parts are molded together.

### CROSS-REFERENCE TO RELATED APPLICATIONS

This application is based on and claims benefit to U.S. provisional applications 62/314,866 filed on Mar. 29, 2016.

### BACKGROUND OF THE INVENTION

The present invention relates to cosmetic and surgical instruments, more specifically to scraping and cleaning instruments also known as curettes. A curette is a medical device used for scraping biological tissue or debris in various medical procedures. Usually, the curette is a small tool with a handle and a tip in a form of a small scoop, loop or gouge. A compound curette usually includes an additional mechanism of delivering substance to the scraping end on the tip of the tool.

Among other things, curettes are used in a biopsy, cleaning procedures or excisions. Curettes are very useful in performing delicate tasks where the access with larger equipment is impracticable or would pose danger to patient.

Particularly, the present invention could be used in cleaning and treating of a toenail a fungus infection, also known as Onychomycosis, affecting 2% to 18% of all people worldwide and 3% to 5% of people in the United States. The likelihood of getting toenail fungus increases with age. Up to 48% of people have at least one affected toe by the time they reach age 70.

Some people consider toenail fungus just a cosmetic problem and don't bother seeking treatment. The treatment procedure for toenail fungus often involves topical application of antifungal agent directly to the affected area. These are sometimes used in combination with systemic drugs. When either or both are used, they are frequently supplemented by scraping of both the fungi and the keratin debris which forms fungi's main feedstock. This scraping has been reported to significantly enhance the treatment. The usual instrument employed for nail debridement comprises an either open or closed cup, spoon, or scoop-shaped curette. The scraping is usually followed with a second step application of the medical agent to the affected area.

### DISCUSSION OF PRIOR ART

As used herein, the term “compound curette” refers to the tool that has a scraping end and a handle containing a substance that can be delivered to the scraping end via the burrow or a channel that connects the handle to the scraping end.

Previously, there were numbers of simple curettes with handles used for one simple function of holding the tool for performing scraping operations. These simple curettes had serious disadvantages, especially if applied in toenail treatment. One of those disadvantages was the rigid nature of the handle. Performing delicate surgical or cosmetic tasks with a tool that was not amenable to the movement of the hand always leads to unwanted movement of the scraping end. In scraping the toenail fungus, such rigidity causes painful sensations for the treated patient. Making the scraping instrument more flexible will undoubtedly decrease painful consequences and improve the accuracy of the process.

Some of the previously described curettes addressed the issue of pathogen proliferation during procedure. In early applications, a glass container with liquid would be supplemented to either dip the curette in or to apply the liquid by means of a separate tool. Such method is inconvenient for treating professional as it requires the use of a second hand to hold the liquid container. Second hand is undoubtedly better used to hold the toenail or any treated area.

U.S. Pat. No. 8,435,189 describes the compound curette that uses a solid handle with a button mechanism, to squeeze the liquid container inserted within, sending the liquid into a burrow towards the scraping end. While achieving the goal of pathogen treatment in one move, the previously described compound curette does not provide the desired flexibility to reach to the contours of the treated area without additional movement of the treating professional's arm. Additionally, the separable reservoir with treating solution was supposed to fit in the hollow chamber of the handle of the previously described curette. Such handle was supposed to provide a mechanism of applying pressure on the separable reservoir to allow the liquid to travel to the scraping end. Such mechanism was usually fitted with the button and additional means of the pressure application. Besides having a rigid inflexible handle, the manufacturing of the described compound curette with the pressure application mechanism is pricey and wasteful. Additionally, not all liquid gets squeezed out, resulting in unneeded waste.

Toenail treatment requires one hand of the treatment provider to steady the toe. The other hand should work fast and with precision, to provide relaxed and painless experience for the patient. Having an inflexible tool makes the treatment uncomfortable for the treating professional and could lead to pain and unwanted damage to the patient.

Thus, there is a clearly felt need for a compound curette which will have the function of delivering a cleaning or medicating liquid directly to the scraping tip to stop pathogen proliferation, be designed to fit comfortably in the curvature of the palm of the treating professional's hand, allowing to treat the patient without injuring his tissues. Such device would be desired in many other surgical and cosmetic applications.

The disclosed invention addresses these and some other issues related to the current state of curette surgical and cosmetics technology.

### SUMMARY OF THE INVENTION

Accordingly, the primary objective of the present invention is to provide a curette which is shaped and designed to allow for comfortable use by the treating professional, while reaching to some curved areas of the treated spot. One of the goals achieved with present invention is a better curette application in scrubbing underneath the affected toenail without injuring the sensitive nail bed, while preventing the pathogen proliferation, by means of a liquid delivery system installed in the soft handle portion of the curette, by delivering the medicated or cosmeceutical solution to the tip portion of the curette.

According to the present invention, medicated solution refers to any over-the-counter or prescription liquid medication that could be used for the treatment in conjunction with cleaning or surgical procedure. For instance, medicated solution could be any liquid topical medication used for the treatment of onychomycosis. Cosmeceuticals are cosmetic products with biologically active ingredients purporting to have medical or drug-like benefits. Cosmeceutical solutions could include, but are not limited to sterilizing liquids, creams, lotions, and ointments.

The proposed curette described in present invention includes a hollow soft and squeezable handle, or a squeezable container, attached to a scraping implement. Squeezable container could be easily compressed by applying pressure either with fingers or a palm of the user's hand. The scraping implement contains a narrow hollow shaft, or a burrow, for delivering the liquid from the container to the scraping tip of the scraping implement. The handle is sized to fit a palm of the hand with the cross-section comfortably fitting inside the hand. One end of the handle could be rounded to provide convenient rest at the base of the hand, close to the wrist. The handle could be made from any squeezable material that could hold the liquid. The shaft and scraping tip could be made from any solid material, to withstand the pressure of application to the treated area.

The liquid from the container could be delivered to the scraping end through the hollow shaft by squeezing the soft handle, creating the pressure inside the container. This liquid could be a saline solution for cleaning a cavity, a solution to soften the keratin debris to facilitate its removal, a disinfectant or antifungal solution, or any other appropriate liquid. This liquid would enter the scoop through small orifice, and could be controlled by adjusting the squeeze pressure on the container-handle, depending on the desire and needs of the user. The liquid flow will also loosen and help eject any slug of material that has been removed by scraping.

The container could be made from a soft natural or synthetic polymer or elastomer, selected from but not limited to polyethylene, polypropylene, polyvinylchloride, polyurethane, Natural polyisoprene, Synthetic polyisoprene, Polybutadiene, Chloroprene rubber, polychloroprene, Neoprene, Baypren, Butyl rubber, Halogenated butyl rubbers, Styrene-butadiene Rubber, Nitrite rubber, Hydrogenated Nitrite Rubbers, EPM (ethylene propylene rubber) and EPDM rubber (ethylene propylene diene rubber), Epichlorohydrin rubber, Polyacrylic rubber, Silicone rubber, Fluorosilicone Rubber, Fluoroelastomers, Perfluoroelastomers, Polyether block amides, Chlorosulfonated polyethylene, Ethylene-vinyl acetate, etc. Softness of the container will allow for the easy pressure generation by simple squeeze of container by fingers or the palm of the user's hand.

The lumen is preferably, but not necessarily, designed such that the opening closest to the reservoir has a smaller diameter and the end connected to the scraping end has a larger opening. This will reduce the likelihood of clogging, because anything that passes through the small end will likely pass all the way through the lumen and into the bowl.

The scraping end is preferably, but not necessarily, round in shape with a scoop in form of a cup, flat scoop, cone, O-ring, or any form desirable for specific application. The bottom part of a scoop is usually round and smooth, designed to prevent injury to a sensitive part of a nail bed. A scraping edge of the scoop could have a round sharp edge allowing for effective scraping of affected areas underneath a toenail. Scoop designed in shape of a cup provides for more efficient liquid containment and delivery to the treated area.

The scraping end could be made of or is coated with an anti-pathogen material, providing an extra safeguard from bacterial or fungal proliferation during the treatment. Such protection could be accomplished by plating the base metal, usually stainless steel, with a metallic surface that possesses anti-bacterial, antiviral, and/or antifungal properties. Such metals could be selected from but are not limited to: silver, copper, zinc, cobalt, nickel, selenium, or compound metals such as copper-containing titanium nitride films, titanium-hafnium, titanium, plus chromium, titanium nitride plus hafnium nitride, titanium nitride plus zirconium nitride, titanium nitride plus chromium nitride or titanium dioxide. The titanium nitride combinations are interesting because titanium nitride also confers anti-corrosion and anti-wear properties when deposited on other surfaces, such as stainless steel. The above materials are presented as examples, and not intended to limit the possible metals or combination of metals that could be used.

Similarly, the scraping end could be made from a hard polymer material and coated with an anti-pathogen agent. The use of plastic would make the curette more economical, providing cheaper disposable instrument. One of the alternative embodiments of the present invention represents a curette molded entirely out of plastic.

The scraping end is connected to the handle-container by a short and hollow tube, also referred as burrow, allowing the transport of liquid from the squeezable handle to a scraping scoop. The tube has a scraping scoop on one end and on the other end it has means to connect to a squeezable handle-container. The scraping end is made of a stainless steel, or any other metal, or a solid polymer material. It is feasible to have a scraping end either removable or permanently attached to a handle-container. Such removable scraping end could be reusable and attachable to a handle-container by means of a screw thread connection or by any other secure connection.

It is a further object of the present invention to provide a corrosion resistant, anti-pathogen coated curette which delivers an antifungal, anti-bacterial, antiviral, or cleaning liquid to the treatment area.

The present invention should not be restricted to the treatment of onychomycosis. It could be used for any other nail ailments as well as for other surgical procedures.

Additional features of the disclosure will become apparent to those skilled in the art upon consideration of the following detailed description of the illustrative embodiment.

### BRIEF DESCRIPTION OF THE DRAWINGS

The detailed description particularly refers to the accompanying figures in which:

FIG. 1 is a prospective view of the compound curette with soft handle and liquid delivery system according to the present invention;

FIG. 2 is a different angle of a prospective view of the compound curette with soft handle and liquid delivery system according to the present invention;

FIG. 3 is a prospective view of the compound curette with soft handle and liquid delivery system with a scraping end detached from a soft squeezable end;

FIG. 4 is a different angle view of the compound curette with soft handle and liquid delivery system with a scraping end detached from a soft squeezable end;

FIG. 5 is a blown-up view of a scraping end from the point of attachment to a squeezable end, and collection of differently shaped scraping utensils;

FIG. 6 is a prospective view of application of the compound curette with soft handle and liquid delivery system for treatment of toenail fungus;

FIG. 7 is a view of an alternative embodiment of the compound curette with soft handle and liquid delivery system according to the present invention.

### DETAILED DESCRIPTION OF PREFERRED EMBODIMENTS

For the purposes of promoting and understanding the principles of the invention, reference will now be made to one or more illustrative embodiments presented in the drawings and specific language will be used to describe the same.

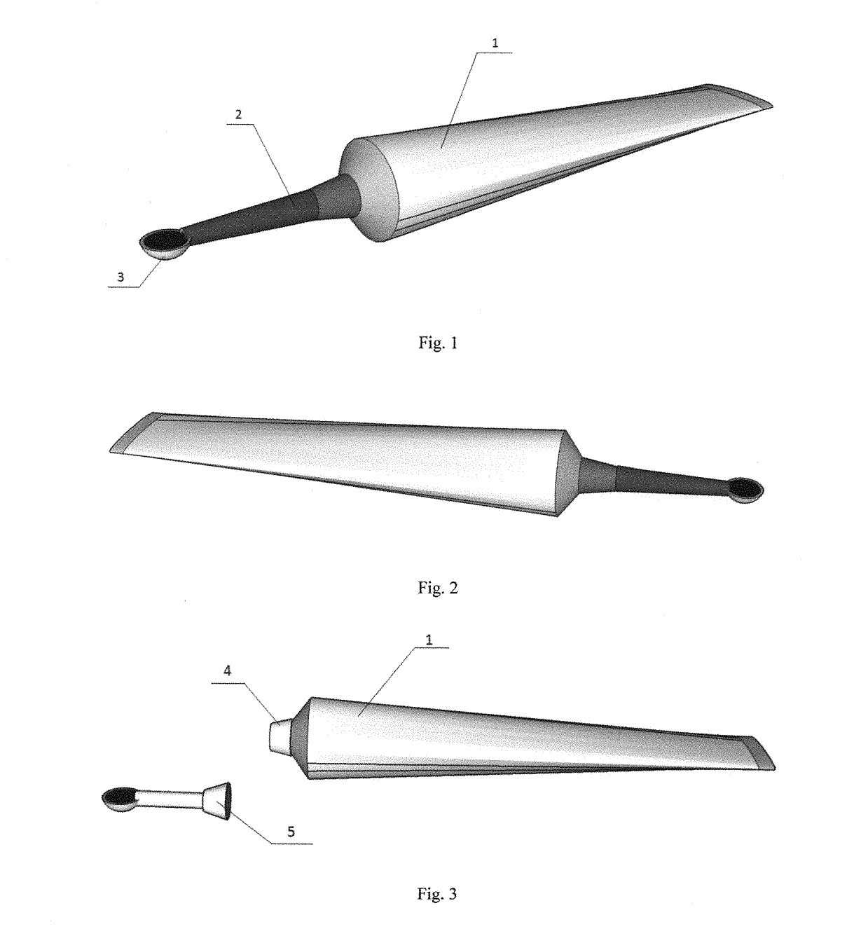

Referring to FIG. 1, a curette with anti-pathogen coating and liquid delivery system includes a soft and squeezable handle 1 made out of a polymer plastic or rubber-like material and hollow inside. The depicted soft and squeezable handle is shaped like a tube to fit comfortably in the palm of the user's hand. The shape of a squeezable handle is not limited to a presented tube, and could be designed in any size or shape for a comfortable use by a treating practitioner. The squeezable handle 1 is attached to a scraping implement (also referred as “scraping end”, or “scraping utensil”), consisting of a solid and hollow tube 2 and a scraping scoop 3. Scraping implement is usually made out of the hard material like metal or hard plastic, and preferably coated with the pathogen inhibiting material selected but not limited to the following: silver, copper, zinc, cobalt, nickel, selenium, or compound metals such as copper-containing titanium nitride films, titanium-hafnium, titanium, plus chromium, titanium nitride plus hafnium nitride, titanium nitride plus zirconium nitride, titanium nitride plus chromium nitride or titanium dioxide or the mixture thereof. Scraping implement could be made entirely out of a pathogen inhibiting material. The soft and squeezable handle is hollow inside and is usually filled with a medicated or cleaning liquid. The tube 2 of the scraping end of the curette is also hollow inside, allowing for the medicated or cleaning liquid to freely travel from the soft and squeezable handle to the scraping scoop or implement 3.

FIG. 2 demonstrates the compound curette with soft handle and liquid delivery system from a different point of view. This view is presented for a better understanding of the present invention.

FIG. 3 demonstrates the embodiment of the improved curette with soft and squeezable handle 1 being detached from the solid end with a scraping utensil. Such ability to detach allows to refill the squeezable handle with a new liquid, and also allows to switch the scraping utensil to a differently designed scraping utensil, allowing for the easy execution of variable tasks. The soft and squeezable handle 1 could be fitted with protruded end 4, designed to fit into the opening 5 of the detachable solid end with a scraping utensil. The protruded end 4 could be secured into the opening 5 by means of a thread or any other latching or clasping mechanism. The soft and squeezable handle demonstrated in the depicted embodiment of the invention is shaped in form of a reservoir, similar to a toothpaste tube. One end of the reservoir is sealed, while the other, protruded end 4 has an opening for the liquid delivery. The present invention is not limited to a presented detachable embodiment. The compound curette could be manufactured as a single unit, when the soft handle is permanently attached to a scraping utensil.

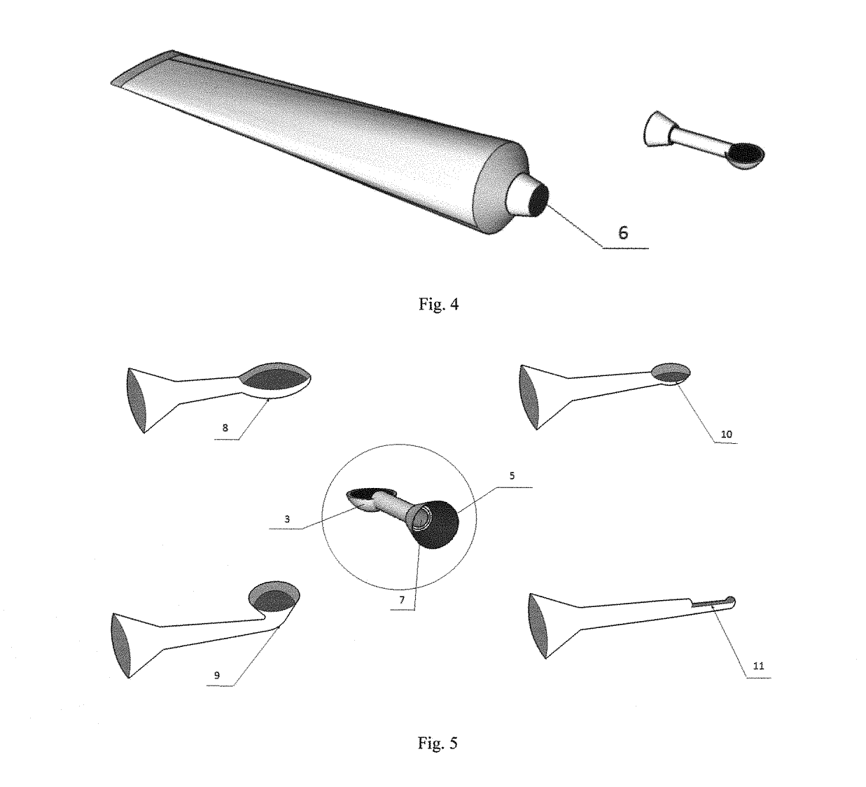

FIG. 4 demonstrates an alternative angle view of the soft and squeezable handle and the detached solid end with a scraping utensil. From this prospective, it could be seen that protruding portion of the soft and squeezable handle has an opening 6, allowing the liquid to be pushed out of the soft and squeezable handle.

A blown-up view of the solid end with a scraping implement could be seen in the middle of the FIG. 5. From the depicted prospective, it could be seen that the solid end's wider opening is designed to fit a protrusion of the soft and squeezable handle. The scraping utensil 3 is connected to the wider opening 5 by means of a tube, with a burrow 7, allowing the liquid to travel from the soft and squeezable handle to the scraping implement 3. Surrounding the blown-up view of the solid end are different embodiments of the scraping implement. Present invention is not meant to be limited only to a toenail treatment. Nevertheless, all of the designs presented have an opening at the top and a smooth bottom. Scoops 8 and 10 are similar and differ mainly in the size of the opening of the cup. Detachable scraping implement 9 ends with a cone-shaped cup, attached to a shaft at the bottom portion. Such implement could be effective in the treatment of common warts. Duct-like design of the implement 11 allows for a deeper penetration and could be very useful in the nail treatment. The scraping end's scoop may be made in form of an O-ring, instead of a cup. Such design would allow for easy rinsing and disposing of the used liquid and debris collected during the treatment. All of the scraping implements according to a present invention are made from either a hard metal, selected from, but not limited to stainless steel, iron, aluminum, silver, copper, zinc, cobalt, nickel and selenium, or a hard polymer material, selected from, but not limited to polypropylene, polyacrylonitrile, polycarbonate, polyvinyl chloride, polyethylene, nylon, Teflon. The base material could be coated with a material that demonstrated a pathogen inhibition. Such coating, combined with a delivery of the medication to the locus of the infection makes the current invention significantly more efficient than previously described curettes.

The compound curette with soft handle and liquid delivery system is intended, among other things, to treat onychomycosis or other nail ailments by removing keratin debris, fungi, and other undesirable materials that may have accumulated in the affected area. The anti-pathogen coating helps to stop or slow the dangerous pathogen proliferation. This feature is greatly important in cosmetic and surgical treatment. Usually, the same curette is used to treat all nails of the patient, requiring frequent sterilization while working from toe to toe. The anti-pathogen coating serves as an additional safeguard, denying the spread of the fungus from an affected nail to a healthy one. A convenient liquid delivery system, manipulated by a simple squeeze of a palm or fingers, allows treating the infection in a previously unreachable area.

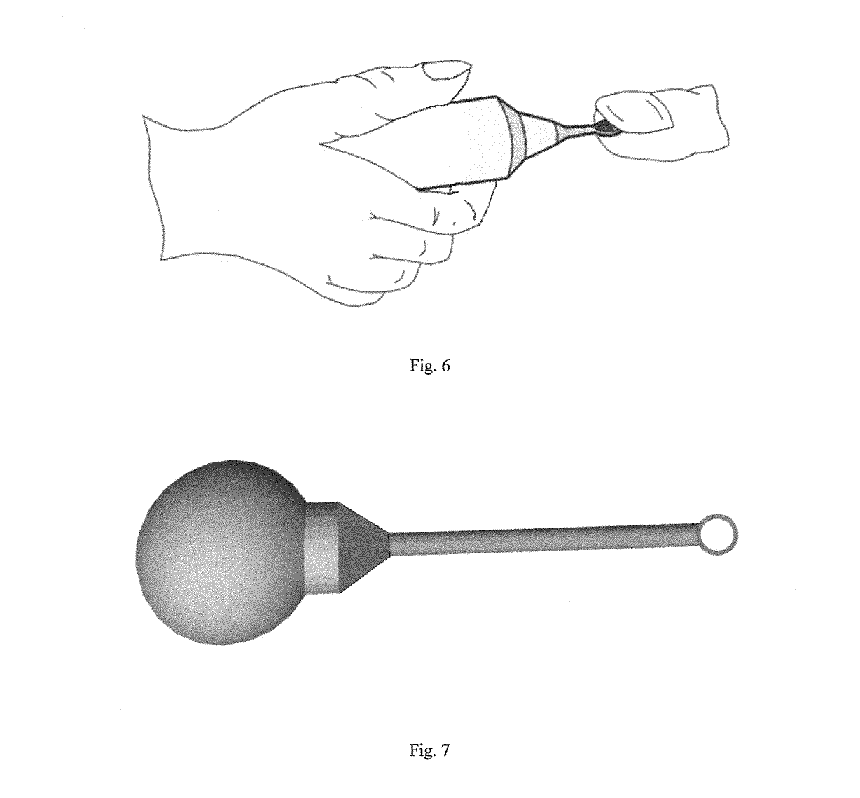

FIG. 6 depicts the intended use, when the practitioner holds the soft and squeezable handle and maneuvers the solid end with scraping implement between the nail and the nail bed of the selected toe or finger. The curette's scraping end is oriented to face upward toward the nail, applying light pressure upward. The curette cup is used to scrape off and scoop away any keratin debris, fungi, and other undesirable materials that may have accumulated in this space. The curette bowl is sometimes rotated in order to facilitate this removal process. As the bowl fills with accumulated debris, it is retracted and emptied. When the procedure is finished, any debris clinging to the curette with layered anti-pathogen protection is mechanically removed by the practitioner or staff, and the curette is sterilized using any of the standard methodologies. While scraping under the affected toe, practitioner may apply pressure on the soft and squeezable handle, sending the medicated or cleaning liquid towards the scraping end of the curette. Such dual function of the suggested invention allows to achieve cleaning and treatment of the affected area in one move, without interruption, reducing the sometimes-unpleasant procedure's duration. The softness of the handle allows the practitioner to have a better grip on the instrument, achieving better maneuverability, and improving the precision of the performed procedure.

FIG. 7 provides a view of an alternative embodiment of the compound curette with soft handle and liquid delivery system according to the present invention. The squeezable handle is made in a spherical shape that would fit entirely in the palm of the user's hand. Such rendition allows for a very easy directional manipulation of the scraping end that is presented on FIG. 7 in shape of an O-ring.

Figures provide preferred embodiment of the invention. However, the invention is not limited to the disclosed configuration. The liquid delivery system could be more complex, allowing for the measured delivery of prescription medication.

While the invention has been illustrated and described in detail in the foregoing drawings and description, the same is to be considered as illustrative and not restrictive in character, it being understood that only illustrative embodiments thereof have been shown and described and that all changes and modifications that come within the sprit and scope of the invention as defined in the following claims are desired to be protected.

1. A compound curette for scraping and delivering liquid to a scraped area comprising:
a squeezable container having an opening;
a scraping utensil having a first end and a second end;
said second end of the scraping utensil shaped for scraping;
a burrow connecting said second end to the first end;
said second end of the scraping utensil being attached at said opening of the squeezable container.

2. The compound curette for scraping and delivering liquid to a scraped area of claim 1, wherein said squeezable container is made from a polymer material selected from the group consisting of polyethylene, polypropylene, polyvinylchloride, polyurethane, Natural polyisoprene, Synthetic polyisoprene, Polybutadiene, Chloroprene rubber, polychloroprene, Neoprene, Baypren, Butyl rubber, Halogenated butyl rubbers, Styrene-butadiene Rubber, Nitrile rubber, Hydrogenated Nitrile Rubbers, EPM (ethylene propylene rubber) and EPDM rubber (ethylene propylene diene rubber), Epichlorohydrin rubber, Polyacrylic rubber, Silicone rubber, Fluorosilicone Rubber, Fluoroelastomers, Perfluoroelastomers, Polyether block amides, Chlorosulfonated polyethylene, Ethylene-vinyl acetate.

3. The compound curette for scraping and delivering liquid to a scraped area of claim 1, wherein said scraping utensil is made of polymer selected from the group consisting of polypropylene, polyacrylonitrile, polycarbonate, polyvinyl chloride, polyethylene, nylon, Teflon.

4. The compound curette for scraping and delivering liquid to a scraped area of claim 1, wherein said scraping utensil is made of metal.

5. The compound curette for scraping and delivering liquid to a scraped area of claim 1, wherein said second end of said scraping utensil is coated with anti-pathogen compound selected from the group consisting of copper-titanium nitride, titanium-hafnium, titanium-chromium, titanium nitride-hafnium nitride, titanium nitride-zirconium nitride, titanium nitride-chromium nitride and titanium dioxide or a mixture thereof.

6. The compound curette for scraping and delivering liquid to a scraped area of claim 1, wherein said scraping utensil is detachable from said squeezable container.

7. The compound curette for scraping and delivering liquid to a scraped area of claim 1, wherein said scraping utensil is permanently attached to the said squeezable container.

8. The compound curette for scraping and delivering liquid to a scraped area of claim 1, wherein said squeezable container is filled with a medicated solution.

9. The compound curette for scraping and delivering liquid to a scraped area of claim 1, wherein said squeezable container is filled with a cosmeceutical solution.

10. A method for scraping tissue samples with a compound curette for scraping and delivering liquid to a scraped area, said method comprising the steps of:
(a) providing a compound curette for scraping and delivering liquid to a scraped area, and fitted with squeezable container filled with a solution;
(b) scraping tissue with a scraping utensil of said compound curette;
(c) applying pressure to said squeezable container to cause said solution to travel to a scraping end of the scraping utensil; and
(d) applying said solution to the scraped tissue.

11. A method of claim 6, wherein said method is used for a treatment of toenail ailments.
